(19) United States
(12) Patent Application Publication
(10) Pub. No.: US 2026/0028679 A1
(43) Pub. Date: Jan. 29, 2026

(54) BIOMARKER FOR DIAGNOSIS OF GASTRIC CANCER

(71) Applicant: SAMSUNG LIFE PUBLIC WELFARE FOUNDATION, Seoul (KR)

(72) Inventors: Jee Yun LEE, Seoul (KR); Min Ae AN, Seoul (KR)

(73) Assignee: SAMSUNG LIFE PUBLIC WELFARE FOUNDATION, Seoul (KR)

(21) Appl. No.: 19/278,199

(22) Filed: Jul. 23, 2025

(30) Foreign Application Priority Data
Jul. 25, 2024 (KR) 10-2024-0098291
Sep. 13, 2024 (KR) 10-2024-0125729

(51) Int. Cl. C12Q 1/6886; C12Q 1/6809; G01N 33/50

(52) U.S. Cl. CPC C12Q 1/6886; C12Q 1/6809; G01N 33/5017; G01N 33/5023; G01N 33/5044; C12Q 2600/118; C12Q 2600/136; G01N 2333/811; G01N 2333/918

(57) ABSTRACT

Proposed are a biomarker for diagnosis of advanced gastric cancer and a use thereof. Specifically, by measuring the level of epithelial cells in which serine peptidase inhibitor, Kunitz type 2 (SPINT2), or post-glycosylphosphatidylinositol (GPI) attachment to proteins 3 (PGAP3) is highly expressed, from a biological sample of a patient, the efficiency in diagnosis of gastric cancer was confirmed to be excellent. Thus, the level of the biomarker can be measured and utilized for prevention, improvement, and/or treatment of gastric cancer.

### CROSS REFERENCE TO RELATED APPLICATION

The present application claims priority to Korean Patent Application Nos. 10-2024-0098291, filed Jul. 25, 2024, and 10-2024-0125729, filed Sep. 13, 2024, the entire contents of each of which is incorporated herein for all purposes by this reference.

### BACKGROUND

### 1. Technical Field

One embodiment of the present disclosure relates to a biomarker for diagnosis of gastric cancer and a use thereof.

### 2. Description of the Related Art

In the discovery process to derive new drug candidates for tumor patients, including those with gastric cancer, decisions on which genes or proteins to target in earlier studies are based on selecting targets highly expressed in tumor tissue by comparing the transcriptomes or proteomes of tumor and normal tissues.

However, there have been the following problems in the related art. First, hospitals and research laboratories have difficulties obtaining normal tissue from cancer patients and thus select biomarkers that are differentially expressed compared to normal tissue of organs, other than gastric cancer from public data, overlooking bias to organ-specific gene expression.

Second, most research laboratories rely on whole-cell RNA-based data extracted without distinguishing diverse tumor cells, stromal cells, and immune cells mixed in the tumor environment. When using the data above to develop drugs, such a developed drug may bind to different types of cells, including tumor cells as well as immune cells, which leads to failure in clinical trials or preclinical stages.

Third, when developing antibody-based drugs, this involves manually searching papers or utilizing databases to determine whether a selected biomarker is expressed on membrane proteins, which is time-consuming (out of approximately 60,000 genes, only approximately 800 genes have been identified as encoding membrane proteins).

Lastly, the immune microenvironment or immunogenicity of patients in which target biomarkers are highly expressed is unknown, making it challenging to predict whether immune-related adverse events will occur when developed as immunotherapeutic agents.

In this regard, the inventors of the present disclosure constructed a pipeline based on transcriptome sequencing data of only patients whose tumors and normal gastric tissues were matched, from a cohort of all advanced gastric cancer patients undergoing endoscopy.

### SUMMARY

One aspect aims to provide a composition for diagnosis of gastric cancer, the composition including a preparation for measuring the level of epithelial cells in which serine peptidase inhibitor, Kunitz type 2 (SPINT2), or post-glycosylphosphatidylinositol (GPI) attachment to proteins 3 (PGAP3) is highly expressed.

Another aspect aims to provide a kit for diagnosis of gastric cancer, the kit including the composition above.

A further aspect aims to provide a method of providing information on diagnosis of gastric cancer, the method including measuring the level of epithelial cells in which SPINT2 or PGAP3 is highly expressed, from a biological sample isolated from an individual.

Yet another aspect aims to provide a method of screening a drug for treatment of gastric cancer, the method including: measuring the level of epithelial cells in which SPINT2 or PGAP3 is highly expressed, from a biological sample isolated; bringing a test substance in contact with the biological sample; measuring the level of the epithelial cells, in which SPINT2 or PGAP3 is highly expressed, from the biological sample after making contact with the test substance; and determining the test substance as a drug for treatment of gastric cancer when the level of the epithelial cells, in which SPINT2 or PGAP3 is highly expressed, decreases within the biological sample.

The inventors of the present disclosure performed analysis using RNA sequencing to identify distinct markers indicating gastric tumors through comparison with normal gastric tissue of advanced gastric cancer patients under various conditions.

The present disclosure provides a composition for diagnosis of gastric cancer, the composition including a preparation for measuring the level of epithelial cells in which SPINT2 or PGAP3 is highly expressed.

In one embodiment of the present disclosure, the composition may further include a preparation for measuring the level of epithelial cells in which one or more factors of syndecan 4 (SDC4), G protein-coupled receptor class C group 5 member A (GPRC5A), a neuronal cell adhesion molecule (NRCAM), and a lipolysis-stimulated lipoprotein receptor (LSR) are highly expressed.

In one embodiment of the present disclosure, the preparation for measuring the level of the epithelial cells may be one or more selected from the group consisting of an oligopeptide, a ligand, a peptide nucleic acid (PNA), an aptamer, and an antibody specifically binding to the epithelial cells in which SPINT2 or PGAP3 is highly expressed.

In one embodiment of the present disclosure, the gastric cancer may be advanced gastric cancer.

In addition, the present disclosure provides a kit for diagnosis of gastric cancer, the kit including the composition.

In addition, the present disclosure provides a method of providing information on diagnosis of gastric cancer, the method including measuring the level of epithelial cells in which SPINT2 or PGAP3 is highly expressed, from a biological sample isolated from an individual.

In one embodiment of the present disclosure, the method may further include determining that locally advanced gastric cancer has developed or is likely to develop when the level of the epithelial cells, in which SPINT2 or PGAP3 is highly expressed, of the biological sample isolated from the individual increases compared to a sample of a normal control group.

In one embodiment of the present disclosure, the biological sample may be whole blood, plasma, serum, sputum, a tear fluid, mucus, a nasal wash, a nasal aspirate, breath, urine, semen, saliva, a peritoneal washing fluid, ascites, a cystic fluid, a meningeal fluid, an amniotic fluid, a glandular fluid, a pancreatic fluid, a lymph fluid, a pleural fluid, a nipple aspirate, a bronchial aspirate, a bronchial washing fluid, a synovial fluid, a joint aspirate, an organ secretion, a cell, a cell extract, or a cerebrospinal fluid.

In addition, the present disclosure provides a method of screening a drug for treatment of gastric cancer, the method including: measuring the level of epithelial cells in which SPINT2 or PGAP3 is highly expressed, from a biological sample isolated; bringing a test substance in contact with the biological sample; measuring the level of the epithelial cells, in which SPINT2 or PGAP3 is highly expressed, from the biological sample after making contact with the test substance; and determining the test substance as a drug for treatment of gastric cancer when the level of the epithelial cells, in which SPINT2 or PGAP3 is highly expressed, decreases within the biological sample.

In one embodiment of the present disclosure, measuring the level of the epithelial cells is to measure the level of the epithelial cells in which SPINT2 or PGAP3 is highly expressed.

According to one aspect, a composition for diagnosis of gastric cancer, the composition including a preparation for measuring the level of epithelial cells in which SPINT2 or PGAP3 is highly expressed, has demonstrated that in terms of advanced gastric cancer, epithelial cells in which SPINT2 or PGAP3 is highly expressed can effectively serve as a biomarker for diagnosis of pathologically advanced gastric cancer. Accordingly, gastric cancer can be effectively diagnosed with the use of the composition. Furthermore, depending on the diagnosis, the epithelial cells in which SPINT2 or PGAP3 is highly expressed can be inhibited, thus enabling effective treatment of gastric cancer.

### BRIEF DESCRIPTION OF THE DRAWINGS

FIG. 1 shows an overview of a drug target discovery pipeline;

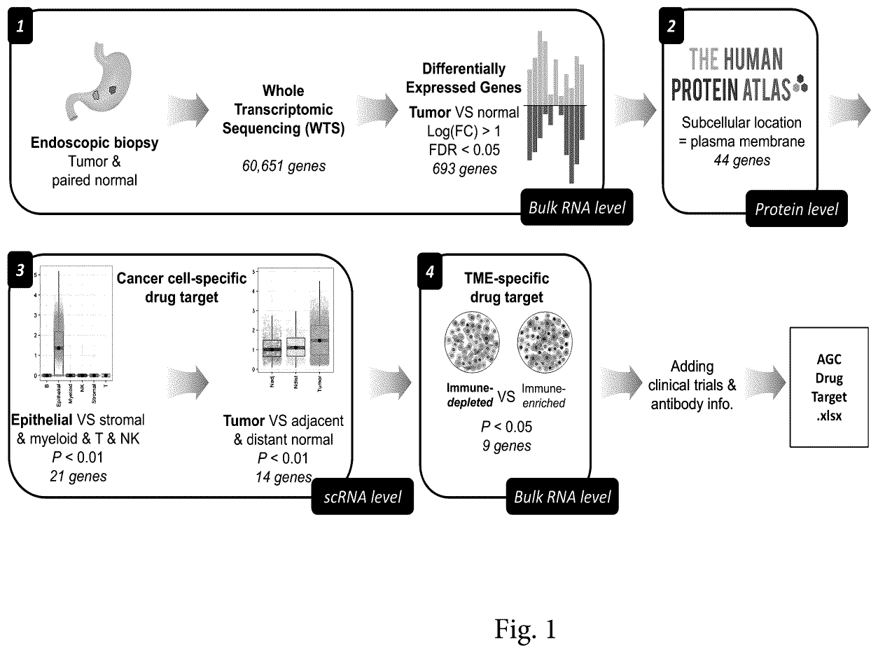

FIG. 2 shows the classification of genes highly expressed in tumor and normal tissues;

FIG. 3 shows expression sites of six genes finally selected, on a Uniform Manifold Approximation and Projection (UMAP);

FIG. 4 shows expression levels of six genes finally selected, in various cell types;

FIG. 5 shows expression levels of six genes finally selected, in epithelial cells of a tumor, a tumor-adjacent normal tissue, and a distant normal tissue; and

FIG. 6 shows a comparison of expression levels of six genes finally selected, in the tumor microenvironment.

### DETAILED DESCRIPTION

The present disclosure provides a composition for diagnosis of gastric cancer, the composition including a preparation for measuring the level of epithelial cells in which serine peptidase inhibitor, Kunitz type 2 (SPINT2), or post-glycosylphosphatidylinositol (GPI) attachment to proteins 3 (PGAP3) is highly expressed.

In the present disclosure, “SPINT2” is a protein known as a serine protease inhibitor, and the expression thereof may increase in gastric tumor epithelial cells.

In the present disclosure, “PGAP3” is a protein involved in the process of GPI anchor biosynthesis, and the expression thereof may increase in gastric tumor epithelial cells.

In the present disclosure, “highly expressed” refers to increased expression of a biomarker compared to a normal control group, which may, for example, imply that the expression of a biomarker of a sample increases by 1.2, 1.3, 1.4, 1.5, 1.8, 2.0, 2.5, 3.0, 5.0, or 10.0 times or more.

In the present disclosure, the “epithelial cell” is a type of cell that forms a cell layer covering the internal organs and surfaces of the body. These cells are densely arranged and perform various functions, including protection, absorption, secretion, and filtration. Epithelial cells are attached onto a thin connective tissue layer called a basement membrane and classified into squamous, cuboidal, and columnar cell types by the shapes thereof. Special structures such as microvilli or cilia may develop on the apical surface of epithelial cells, enhancing cell functions.

In one embodiment of the present disclosure, examples of methods of measuring or comparing and analyzing the level of epithelial cells may include protein chip analysis, immunoassay, ligand binding assay, matrix-assisted laser desorption/ionization time-of-flight (MALDI-TOF) mass spectrometry, surface-enhanced laser desorption/ionization time-of-flight (SELDI-TOF) mass spectrometry, radioimmunoassay, radial immunodiffusion, Ouchterlony radial immunodiffusion, rocket immunoelectrophoresis, tissue immunostaining, complement fixation assay, two-dimensional electrophoresis analysis, liquid chromatography-mass spectrometry (LC-MS), liquid chromatography-tandem mass spectrometry (LC-MS/MS), Western blotting, enzyme-linked immunosorbent assay (ELISA), and the like, but are not limited thereto.

In one embodiment of the present disclosure, the composition may further include a preparation for measuring the level of epithelial cells in which one or more factors of syndecan 4 (SDC4), G protein-coupled receptor class C group 5 member A (GPRC5A), a neuronal cell adhesion molecule (NRCAM), and a lipolysis-stimulated lipoprotein receptor (LSR) are highly expressed.

In the present disclosure, the “SDC4” is a cell surface protein involved in various cell functions, including cell signaling, cell adhesion, and cell migration, and the expression thereof may increase in gastric tumor epithelial cells.

In the present disclosure, the “GPRC5A” is a protein involved in various tumor-related signaling pathways, including the cyclic adenosine monophosphate (cAMP), nuclear factor kappa B (NF-κB), signal transducer and activator of transcription 3 (STAT3), and focal adhesion kinase/Src (FAK/Src) signaling pathways, and the expression thereof may increase in gastric tumor epithelial cells.

In the present disclosure, the “NRCAM” is a protein involved in processes, including axon guidance, neuronal migration, and synapse formation, and the expression thereof may increase in gastric tumor epithelial cells.

In the present disclosure, the “LSR” is a lipolysis-stimulated lipoprotein receptor protein, and the expression thereof may increase in gastric tumor epithelial cells.

In one embodiment of the present disclosure, the preparation for measuring the level of the epithelial cells may be one or more selected from the group consisting of an oligopeptide, a ligand, a peptide nucleic acid (PNA), an aptamer, and an antibody specifically binding to the epithelial cells in which SPINT2 or PGAP3 is highly expressed.

The term “antibody” as used herein refers to a substance that specifically binds to an antigen and causes an antigen-antibody reaction. For the purpose of the present disclosure, the antibody may be an antibody that binds to macrophages in which interleukin-1 beta (IL-1ß) is highly expressed. The antibody of the present disclosure includes all polyclonal antibodies, monoclonal antibodies, and recombinant antibodies. The antibody may be prepared easily using techniques widely known in the related art. In addition, the antibody of the present disclosure may include not only a complete form having two full-length light chains and two full-length heavy chains, but also a functional fragment of an antibody molecule. The functional fragment of an antibody molecule refers to a fragment having at least an antigen-binding function, and examples thereof include Fab, F(ab′), F(ab′)₂, Fv, and the like.

The term “PNA” as used herein, referring to an artificially synthesized polymer similar to DNA or RNA, was first introduced in 1991 by Professors Nielsen, Egholm, Berg, and Buchardt of the University of Copenhagen, Denmark. While DNA has a backbone composed of phosphoric acid and ribose sugar, PNA is composed of repeating units of N-(2-aminoethyl)-glycine linked by peptide bonds and, therefore, is used in molecular biology, diagnostic analysis, and antisense therapy due to significantly improved stability and binding affinity to DNA or RNA.

The term “aptamer” as used herein is an oligonucleotide or peptide molecule and refers to a ligand-specific DNA or RNA molecule having high affinity to proteins. Aptamers refer to single-stranded DNA or RNA having a specific binding capability to a particular substance and naturally have a unique tertiary structure. Aptamers can be mass-produced at low costs in a short time using chemical synthesis techniques and hardly involve batch-to-batch variability, which is remarkably advantageous in terms of productivity. In addition, due to being highly stable against changes in the surrounding environment, such as pH or temperature changes, the potential use of aptamers in various fields, including the detection of target substances and the development of diagnostic sensors for diseases, has recently been highly regarded.

In one embodiment of the present disclosure, the gastric cancer may be advanced gastric cancer. In this case, the term “advanced gastric cancer”, indicating the later stage of gastric cancer, refers to a condition where cancer cells have infiltrated deeper into the tissue layer beyond the gastric mucosa or have metastasized to the peripheral lymph nodes or other organs.

In the present disclosure, the “diagnosis” includes determining the susceptibility of a subject to a particular disease or condition, determining whether a subject currently has a particular disease or condition, determining the prognosis (for example, identifying pre-metastatic or metastatic cancer status, determining the stage of cancer, or determining the response of cancer to treatment) of a subject suffering from a particular disease or condition, or therametrics (for example, monitoring the condition of an individual to provide information on the efficacy of treatment). For the purpose of the present disclosure, the diagnosis is to determine whether the condition has developed or there is a possibility (risk) of developing the condition, the stage of the cancer, or the survival rate or treatment response of cancer patients.

In the present disclosure, the “stage” refers to the extent to which cancer cells have spread or the stage of cancer progression. The international classification of cancer by progression is typically based on the TNM staging classification. In this case, “T (tumor Size)” refers to a classification based on the size of the primary tumor, “N (lymph node)” refers to a classification based on the degree of lymph node metastasis, and “M (metastasis)” refers to a classification based on whether cancer has metastasized to other organs.

In addition, the present disclosure provides a kit for diagnosis of gastric cancer, the kit including the composition.

In addition, the present disclosure provides a method of providing information on diagnosis of gastric cancer, the method including measuring the level of epithelial cells in which SPINT2 or PGAP3 is highly expressed, from a biological sample isolated from an individual.

The term “individual” as used herein means all living organisms including a human, as well as a mouse, livestock, and the like. Specific examples thereof may include mammals, including humans.

In one embodiment of the present disclosure, the method may further include determining that advanced gastric cancer has developed or is likely to develop when the level of the epithelial cells, in which SPINT2 or PGAP3 is highly expressed, of the biological sample isolated from the individual increases compared to a sample of a normal control group.

In one embodiment of the present disclosure, the biological sample may be whole blood, plasma, serum, sputum, a tear fluid, mucus, a nasal wash, a nasal aspirate, breath, urine, semen, saliva, a peritoneal washing fluid, ascites, a cystic fluid, a meningeal fluid, an amniotic fluid, a glandular fluid, a pancreatic fluid, a lymph fluid, a pleural fluid, a nipple aspirate, a bronchial aspirate, a bronchial washing fluid, a synovial fluid, a joint aspirate, an organ secretion, a cell, a cell extract, or a cerebrospinal fluid.

In addition, the present disclosure provides a method of screening a drug for treatment of gastric cancer, the method including: measuring the level of epithelial cells in which SPINT2 or PGAP3 is highly expressed, from a biological sample isolated; bringing a test substance in contact with the biological sample; measuring the level of the epithelial cells, in which SPINT2 or PGAP3 is highly expressed, from the biological sample after making contact with the test substance; and determining the test substance as a drug for treatment of gastric cancer when the level of the epithelial cells, in which SPINT2 or PGAP3 is highly expressed, decreases within the biological sample.

The term “treatment” as used herein may mean any act that results in improvement or alleviation of gastric cancer symptoms in an individual by administration of a pharmaceutical composition according to one aspect.

In one embodiment of the present disclosure, measuring the level of the epithelial cells is to measure the level of the epithelial cells in which SPINT2 or PGAP3 is highly expressed.

Hereinafter, the present disclosure will be described in more detail through examples. However, the following examples are disclosed for illustrative purposes of the present disclosure, and the scope of the present disclosure is not limited by the following examples.

### EXAMPLE

### 1. Experimental Method

### (1) Sample Collection

All primary tissues and paired normal tissues were collected from patients who underwent endoscopic biopsy. After pathologic evaluation, when tumor purity was confirmed to be 40% or higher, tissue RNA was extracted for transcriptome sequencing using the QIAamp Mini Kit (Qiagen, Hilden, Germany) according to the manufacturer's instructions. After measuring concentration and 260/230 nm ratio using the ND1000 spectrophotometer (Nanodrop Technologies, Thermo-Fisher Scientific), RNA was quantified using the Qubit fluorometer (Life Technologies).

### (2) Whole-Transcriptome Sequencing

According to the manufacturer's protocol, a sequencing library was constructed from 100 ng of total RNA using the TruSeq RNA Access library preparation kit (Illumina, San Diego, CA, USA). Total RNA was first fragmented into small pieces using divalent cations at a high temperature. The cleaved RNA fragments were transcribed into first-strand cDNA using the SuperScript II reverse transcriptase (Invitrogen, #18064014) and random primers. Then, the resulting product was purified and enriched by a polymerase chain reaction (PCR) to produce a cDNA library. The pooled library was incubated with a biotinylated oligo cocktail corresponding to the coding regions of the genome. Library molecules were captured via a hybridized biotinylated oligo probe using streptavidin-conjugated beads. After two rounds of hybridization/capture reactions, the enriched library molecules were applied to a second round of PCR amplification. The captured library was quantified using the KAPA Library Quantification Kit for Illumina sequencing platforms according to the quantitative PCR (qPCR) Quantification Protocol Guide (KAPA BIOSYSTEMS, #KK4854) and evaluated using the TapeStation D1000 ScreenTape (Agilent Technologies, #5067-5582). The indexed library was then submitted to the Illumina Novaseq 6000 (Illumina, Inc., San Diego, CA, USA) to perform paired-end (2×100 bp) sequencing.

The paired-end output was annotated with Ensembl (version 98), and annotated RNA sequence reads were matched to the human reference genome (GRCh38) using STAR (version 2.6.1). Gene expression was quantified in transcripts per million (TPM) using RNA-Seq by Expectation-Maximization (RSEM) (version 1.3.1) with optional parameters set as suggested by the Genotype-Tissue Expression (GTEx) project.

### (3) Analysis of Differential Expression Genes (DEGs)

The TPM of all 60,651 genes was normalized, and genes with a TPM sum of less than 20 for all patients were excluded. Using the edgeR algorithm, 693 genes statistically upregulated in tumor tissue were discovered.

### (4) Biomarker Classification and Genes Encoding Cell Membrane Proteins

The 693 genes were mapped to subcellular locations using the Protein Atlas database. As a result, only 44 genes were classified as encoding cell membrane proteins.

### (5) Single-Cell RNA Sequencing

For single-cell preparation, tumor tissue was dissociated using the gentleMACS Dissociator and the Tumor Infiltrating Lymphocyte Kit (Miltenyi Biotec) according to the manufacturer's protocols. The cells were then frozen and preserved in liquid nitrogen until use. All samples demonstrated an average survival rate of approximately 90% after thawing. Next, 5′ gene expression profiling was performed on the same single-cell suspensions using the Chromium Single Cell V (D) J Solution from 10× Genomics according to the manufacturer's instructions. Up to 8,000 cells were loaded onto a 10× Genomics cartridge for each sample. A 5′ gene expression library with cell barcodes attached was constructed and sequenced to a depth of approximately 50,000 reads per cell using the NovaSeq 6000 platform (Illumina).

### (6) Preprocessing and Annotation

The scRNA-seq reads were aligned to GRCh38, the human genome reference, and quantified using Cellranger (version 5.0). Data for all samples were combined in R v.4.1 using the Seurat Package v.4.0. Doublets were subjected to filtration using Scrublet. In addition, cells with a high mitochondrial read ratio (>30%) and low-quality libraries (<400 genes) were subjected to filtration. After performing normalization, scaling, and principal component analysis on the data for each sample, batch correction was carried out using Harmony. The dimensionality was reduced using the Uniform Manifold Approximation and Projection (UMAP) algorithm for visual representation, and cell clusters were identified using the shared nearest neighbor modularity optimization-based clustering algorithm. Various cell type clusters for each cluster were identified using the “FindAllMarkers” function in Seurat and annotated on the basis of the expression of representative lineage markers.

### 2. Experimental Results

### (1) Analysis Results of Bulk RNA Expression Level

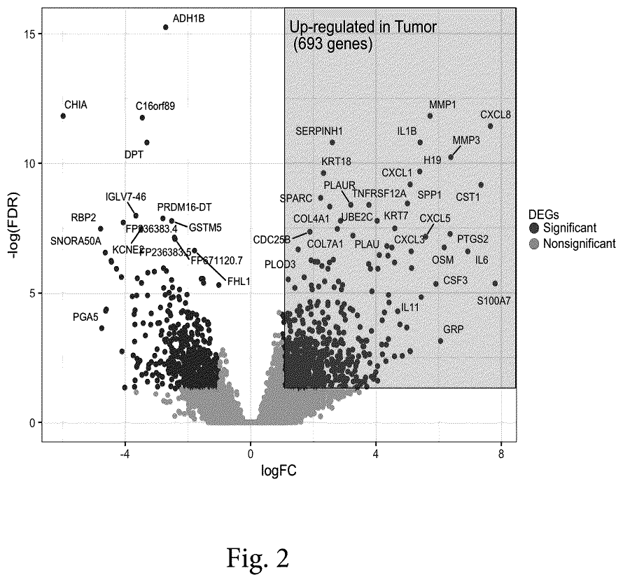

When analyzing the expression level of bulk RNA (FIG. 2), genes highly expressed in tumor tissue (right) and normal tissue (left) were classified, and genes significantly highly expressed statistically were marked in purple. From the tumor tissue, 693 genes significantly highly expressed were extracted.

### (2) Cell Phenotype Annotation

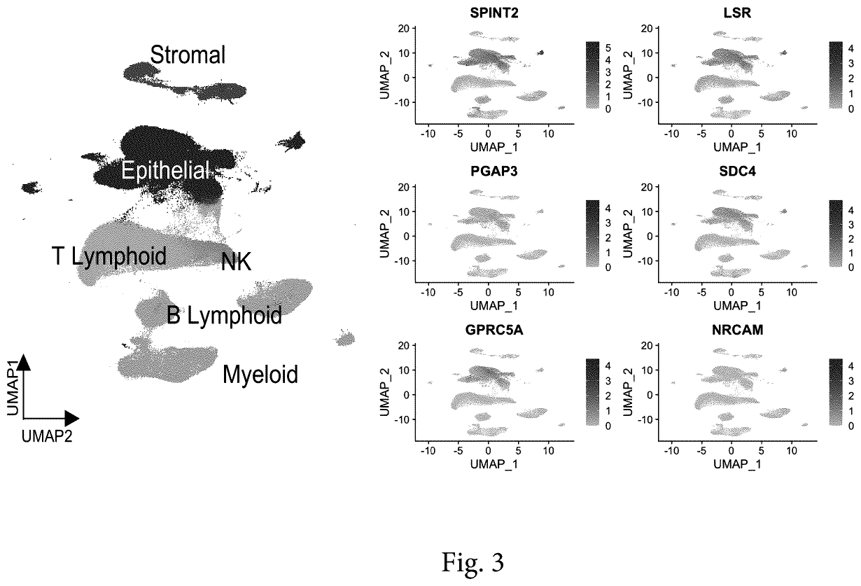

The types of epithelial cells, stromal cells, and four types of immune cells (T lymphocytes, natural killer (NK) cells, B lymphocytes, and myeloid cells) were labeled, and the expression of target genes was identified on the UMAP graph (FIG. 3). FIG. 3 only shows the biomarkers of the present disclosure out of the 693 genes significantly highly expressed in the tumor tissue.

### (3) Analysis of Target Markers Specifically Expressed in Epithelial Cells

When analyzing the expression levels from the single-cell transcriptome data where cell types were identified, multiple comparisons were conducted using post-hoc statistical tests to extract genes specifically highly expressed in epithelial cells, and targets satisfying p<0.05 were selected.

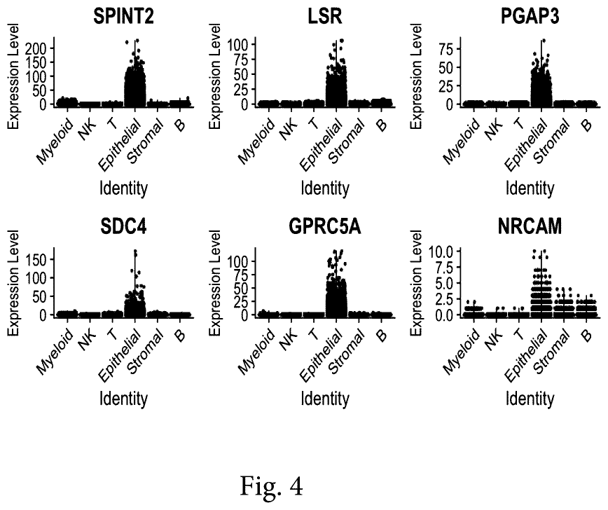

As a result, 21 targets were extracted. The analysis results are shown only for the finally selected targets (FIG. 4).

### (4) Analysis of Target Markers Highly Expressed in Tumor

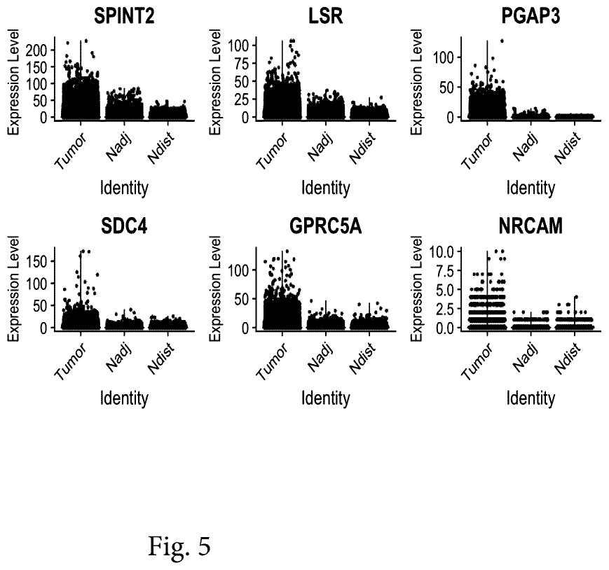

After performing endoscopic biopsy on different locations, cell types were identified through single transcriptome sequencing, and expression in epithelial cells was compared by tissue location. By comparing the expression levels in the epithelial cells of a tumor, a tumor-adjacent normal tissue, and a distant normal tissue through multiple comparisons using post-hoc statistical tests, targets specifically highly expressed in the tumor location and statistically meaningful (p<0.05) were selected (FIG. 5). As a result, 14 targets were extracted. FIG. 5 only shows the analysis results for the finally selected targets.

(5) Selection of Targets Highly Expressed in Patient Group with Low Immunogenicity

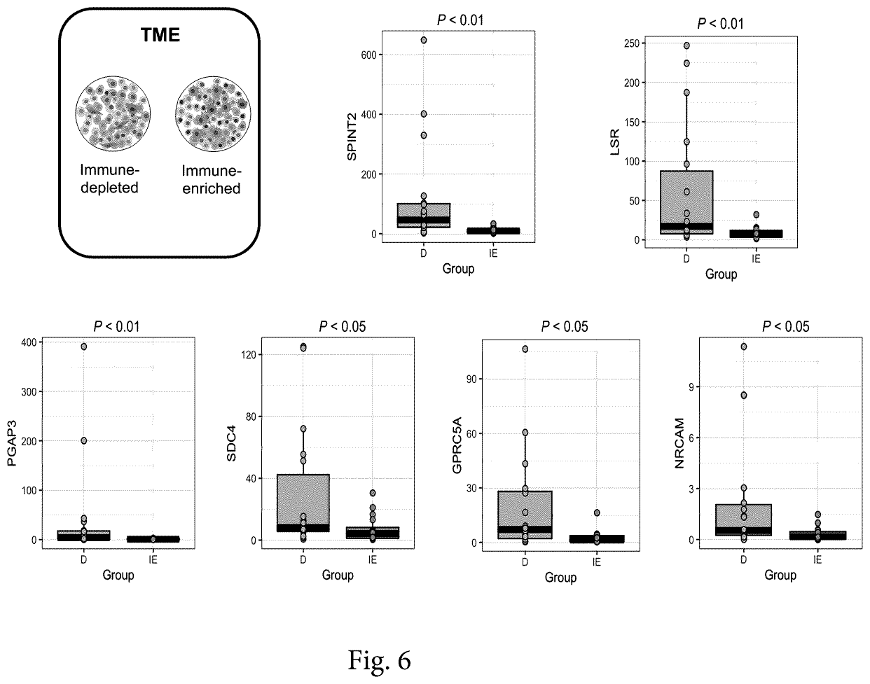

Bulk RNA sequencing-derived TPM values were analyzed utilizing the molecular functional portrait (MFP) algorithm to predict the tumor microenvironment, which was then classified into immune cell-depleted (D) and immune cell-enriched (IE) environments. Once candidate targets are highly expressed in patients with high immunogenicity, immune-related adverse events may occur when developed as T-cell engagers, such as bispecific T-cell engager (BiTE) targeting both tumor and immune cells. For this reason, to help predict such potential adverse events, the Mann-Whitney U test based on immunogenicity was conducted to select nine targets with significantly high immunogenicity from a patient group. Of all these, six markers were finally selected (FIG. 6).

From the above results, it is seen that the epithelial cells, in which SPINT2 or PGAP3 is highly expressed, of the present disclosure can usefully serve as a marker for diagnosis of gastric cancer, particularly, advanced gastric cancer.

What is claimed is:

1. A method of providing information on diagnosis of gastric cancer, the method comprising:
measuring a level of epithelial cells in which factor of SPINT2 or PGAP3 is highly expressed, from a biological sample isolated from an individual.

2. The method of claim 1, wherein the factor further comprising one or more factors of syndecan 4 (SDC4), G protein-coupled receptor class C group 5 member A (GPRC5A), a neuronal cell adhesion molecule (NRCAM), and a lipolysis-stimulated lipoprotein receptor (LSR).

3. The method of claim 1, further comprising:
determining that advanced gastric cancer has developed or is likely to develop when the level of the epithelial cells, in which factor is highly expressed, of the biological sample isolated from the individual increases compared to a sample of a normal control group.

4. The method of claim 1, wherein the biological sample is whole blood, plasma, serum, sputum, a tear fluid, mucus, a nasal wash, a nasal aspirate, breath, urine, semen, saliva, a peritoneal washing fluid, ascites, a cystic fluid, a meningeal fluid, an amniotic fluid, a glandular fluid, a pancreatic fluid, a lymph fluid, a pleural fluid, a nipple aspirate, a bronchial aspirate, a bronchial washing fluid, a synovial fluid, a joint aspirate, an organ secretion, a cell, a cell extract, or a cerebrospinal fluid.

5. The method of claim 1, wherein the gastric cancer is advanced gastric cancer.

6. A method of screening a drug for treatment of gastric cancer, the method comprising:
measuring a level of epithelial cells in which factor of SPINT2 or PGAP3 is highly expressed, from a biological sample isolated;
bringing a test substance in contact with the biological sample;
measuring a level of the epithelial cells, in which factor is highly expressed, from the biological sample after making contact with the test substance; and
determining the test substance as a drug for treatment of gastric cancer when the level of the epithelial cells, in which factor is highly expressed, decreases within the biological sample.

7. The method of claim 6, wherein the factor further comprising: one or more factors of syndecan 4 (SDC4), G protein-coupled receptor class C group 5 member A (GPRC5A), a neuronal cell adhesion molecule (NRCAM), and a lipolysis-stimulated lipoprotein receptor (LSR).

8. The method of claim 6, wherein the measuring of the level of the epithelial cells is to measure the level of the epithelial cells in which factor is highly expressed.

9. The method of claim 6, wherein the gastric cancer is advanced gastric cancer.
